(19) United States
(12) Patent Application Publication
(10) Pub. No.: US 2023/0203413 A1
(43) Pub. Date: Jun. 29, 2023

(54) Pediococcus acidilactici for Promoting Production of Flavor Compounds in Fermented Food and Its Application

(71) Applicant: Jiangnan University, Wuxi (CN)

(72) Inventors: Qun WU, Wuxi (CN); Yan Xu, Wuxi (CN); Guanyi Qu, Wuxi (CN)

(21) Appl. No.: 18/068,584

(22) Filed: Dec. 20, 2022

(30) Foreign Application Priority Data
Feb. 25, 2022 (CN) 202210175390.0

(51) Int. Cl. C12G 3/026; C12N 1/20

(52) U.S. Cl. CPC C12G 3/026; C12N 1/205; C12R 2001/01

(57) ABSTRACT

The disclosure provides P. acidilactici for promoting production of flavor compounds in a fermented food and its application, which belongs to the fields of microorganism, bioengineering technology, and foods. The P. acidilactici with a accession number of CGMCC NO. 22237 obtained in the disclosure is screened from fermented grains of Baijiu, has strong acid resistance, ethanol resistance high temperature resistance, and high salinity resistance can adapt to production environments for a plurality of foods; it has high capability of producing alcohols, acids and esters type flavor compounds; it has high substrate utilization rate and does not have a gene for encoding enzymes in the synthesis pathway of biogenic amines or nitrites. By being co-fermented with S. cerevisiae, P. kudriavzevii, L. acetotolerans, or a brewed food flavor microorganism starter culture, the strain can promote production of flavor compounds, and can be applied to foods and beverages such as pickles, enzymes and fermented meat products to improve the flavor characteristics of the fermented foods, and decrease the production cost.

The instant application contains a Sequence Listing in XML format as a file named “YGHY-2022-68 SEQ.xml”, created on Nov. 14, 2022, of 3.44 kB in size, and which is hereby incorporated by reference in its entirety.

### TECHNICAL FIELD

The disclosure relates to P. acidilactici for promoting production of flavor compounds in a fermented foods and an application thereof, and belongs to the fields of microorganism, bioengineering technology, and foods.

### BACKGROUND

As a quite important industry, fermented foods affect human life in a variety of aspects, such as economy and health. One of the important reasons why fermented foods are popular among consumers is that fermented foods contain a variety of flavor compounds. Most of the compounds are produced with the growth and metabolism of microorganisms. Among the variety of microorganisms, lactic acid bacteria play an important role. Pediococcus is common lactic acid bacteria genus, widely exists in different food brewing systems and environments and has a high relative abundance in different fermented foods. Current research has shown that lactic acid bacteria mainly produce acids in fermentation process. However, there are great differences in characteristics of physical and chemical metabolism, flavor metabolism and safety among lactic acid bacteria strains. Thus, it is urgent to screen desirable strains to improve the overall characteristics of fermented foods.

There are still challenges in screening lactic acid bacteria strains as the following reasons. (1) A fermented food system is typically a high-acidity, high-alcohol, high-temperature and high-salinity environment that requires more microorganisms with strong stress tolerance. (2) The microbiota requires strains with high metabolic activity, high metabolic diversity, but it is challenging to screen the strains that could produce various flavor compounds with high yields. (3) Microbial interaction exists widely, and it is necessary to screen strains that could regulate or promote other microorganisms to produce flavor compounds. (4) Food-safety strains are required to be screened to ensure food safety. (5) The industry of fermented foods needs to upgrade to a more environmentally and economically friendly direction, thus, strains with high substrate utilization should be screened. At present, the research on lactic acid bacteria in a food fermentation system mainly focuses on Lactobacillus strains, and there is a small number of research and application of other types of lactic acid bacteria. Pediococcus exists in fermentation processes of a plurality of foods, but there is a lack of in-depth understanding of its functions such as flavor compound characteristics, tolerance, interaction with other microorganisms, safety, and substrate utilization in food fermentation. It is a feasible and promising method to improve product quality by screening and applying Pediococcus in food brewing.

### SUMMARY

According to the disclosure, a P. acidilactici strain with desirable fermentation characteristics was screened from fermented grains in fermentation of Baijiu (Chinese liquor). The strain not only has the capability of high production of flavor compounds, but also has good stress tolerance. Moreover, the strain possesses food safety characteristics, and can promote growth and metabolism of other functional microorganisms. Therefore, the strain is an excellent strain, beneficial to production and application of food fermentation. It can be used for single-bacterium or multi-bacterium preparations, and is safe to be applied to food fermentation and to improve food quality.

The disclosure provides a P. acidilactici, and the P. acidilactici was deposited in China General Microbiological Culture Collection Center on Apr. 25, 2021, addressing in 3 #, No. 1 Courtyard, Beichen West Road, Chaoyang District, Beijing, with an accession number of CGMCC NO. 22237.

In an implementation, the P. acidilactici is screened and identified from fermented grains of Baijiu, and has strong acid resistance, ethanol resistance, high temperature resistance and salt resistance, as well as high flavor compound production, high substrate utilization rate, and safety characteristics of reducing potential harmful substances such as biogenic amines and nitrites.

The disclosure further provides a microbial preparation containing the P. acidilactici CGMCC NO. 22237.

In an implementation, the microbial preparation includes a liquid preparation or a solid preparation, and the P. acidilactici in the microbial preparation has a viable content ranging from 10⁸-10¹⁰ CFU/mL or CFU/g.

In an implementation, the microbial preparation includes but is not limited to Daqu (starter), Xiaoqu (starter), Maiqu (starter made of wheat), Fuqu (starter made of bran), Jiuyao (starter) or other types of microbial preparations using the P. acidilactici CGMCC NO. 22237.

In an implementation, the solid preparation is a solid inoculant containing the P. acidilactici CGMCC NO. 22237, and a preparation method of the solid inoculant includes the following step: inoculating a P. acidilactici CGMCC NO. 22237 culture into a solid medium and culturing for 30-48 h to obtain a solid bran inoculant with a water content of 5-10% and a viable number ranging from 10⁸-10¹⁰ CFU/g. The solid medium is prepared by wetting brans or corn flour with water and then steam them for 20-40 min.

The disclosure further provides a method for co-culturing the P. acidilactici CGMCC NO. 22237 with Saccharomyces cerevisiae, Pichia kudriavzevii, Lactobacillus acetotolerans and a complex starter culture.

In an implementation, the co-culturing refers to culture the P. acidilactici CGMCC NO. 22237 mixed with the S. cerevisiae, the P. kudriavzevii, or the L. acetotolerans according to a ratio of (10⁵-10⁶) CFU/g:(10⁶-10⁷) CFU/g. The P. acidilactici CGMCC NO. 22237 and the complex starter culture are cultured after being mixed according to a ratio of (10⁵-10⁶) CFU/g:(10⁶-10⁷) CFU/g.

In an implementation, the co-culturing is carried out by sealed fermentation at 28-37° C. for 5-7 d.

The P. acidilactici CGMCC NO. 22237 is a flavor compound production promoting microorganism that is capable of promoting the S. cerevisiae, the P. kudriavzevii, the L. acetotolerans, and starter culture to generate flavor compounds.

A P. acidilactici CGMCC NO. 22237 inoculant or a microbial preparation containing the P. acidilactici CGMCC NO. 22237 is applied in Baijiu production for improvement of flavor characteristics and quality of Baijiu.

In an implementation, the P. acidilactici CGMCC NO. 22237 inoculant is enhanced to a Baijiu fermentation system to promote the production of flavor compounds.

The disclosure further provides an application of the P. acidilactici CGMCC NO. 22237 in fermented foods and fermented beverages.

In an implementation, the fermented foods include but are not limited to fermented pickles, fermented sausages, fermented meat products, fermented fish, fermented shrimp paste, fermented bean paste, and fermented cheese.

In an implementation, the fermented beverages include but are not limited to enzymes.

The disclosure further provides an application of the P. acidilactici CGMCC NO. 22237 or the microbial preparation containing the P. acidilactici CGMCC NO. 22237 in Baijiu fermentation.

The disclosure further provides an application of the P. acidilactici CGMCC NO. 22237 or the microbial preparation containing the P. acidilactici CGMCC NO. 22237 in fermented foods.

### Beneficial Effects

In the disclosure, the screened P. acidilactici CGMCC NO. 22237 from the fermented grains of Baijiu, has the properties of acid resistance, ethanol resistance, high temperature resistance and salt resistance. The strain can grow in an environment with a pH value as low as 2.1, or with an ethanol concentration as high as 12% (v/v), or at a temperature as high as 50° C., or with a salinity as high as 130 g/L, and can adapt to fermentation environments for different foods. The strain has good capability of producing alcohols, acids, esters and other types of flavor compounds with a total yield as high as 1.19 mg/kg, and can also improve flavor compound production of the S. cerevisiae, the P. kudriavzevii, the L. acetotolerans or the starter culture. During co-fermentation by P. acidilactici CGMCC NO. 22237 with S. cerevisiae, P. kudriavzevii, L. acetotolerans or the starter culture can have higher total production of flavor compounds than that of independent use of the above microbial inoculant or that of the complex starter culture. The P. acidilactici strain has genes for encoding a plurality of substrate degrading enzymes, so that it is potential to utilize a plurality of substrates, so that substrate utilization can be improved. In addition, the P. acidilactici strain has no genes encoding enzymes involved in synthesis of biogenic amines or nitrites, reducing the production of biogenic amines or nitrites, and therefore, the P. acidilactici strain is a food safety strain. In conclusion, it is an excellent, safe brewing and a flavor compound production promoting strain. The P. acidilactici can be applied to fermentation of Baijiu, pickles, enzymes, fermented meat products and it's potential to improve the flavor characteristics of the quality of fermented foods and decrease the production cost.

Collection of Biological Materials

P. acidilactici PA624, classified as P. acidilactici, was deposited in China General Microbiological Culture Collection Center on Apr. 25, 2021, addressing at 3 #, No. 1 Courtyard, Beichen West Road, Chaoyang District, Beijing, with an accession number of CGMCC NO. 22237.

### DESCRIPTION OF FIGURES

FIG. 1 shows gene distribution of P. acidilactici CGMCC NO. 22237 annotated in KEGG database.

FIG. 2 shows flavor compound production of the P. acidilactici CGMCC NO. 22237 inoculant co-cultured with other microbial preparations.

### DETAILED DESCRIPTION

Culture Medium:

An MRS solid medium includes the following components: 5 g/L of beef extract, 10 g/L of peptone, 4 g/L of yeast extract, 0.2 g/L of MgSO₄·7H₂O, 5 g/L of NaCl, and 1 g/L of Tween 80, the components were sterilized at a pH of 7.0 for 20 min, and then the medium was poured into a culture dish.

A basic medium includes the following components: 5 g/L of beef extract, 10 g/L of peptone, 4 g/L of yeast extract, 0.2 g/L of MgSO₄ 7H₂O, 5 g/L of NaCl, and 1 g/L of Tween 80, and the components were sterilized at a pH of 7.0 for 20 min.

A solid medium was prepared by wetting raw brans or corn flour with water and then steaming for 30 min.

A liquid grain medium is prepared as follows: sorghum or wheat raw material samples were crushed and mixed with water; then 30-50 U/g of high-temperature α-amylase was added to the mixture and steamed for 1-3 h; 10-50 U/g of a glucoamylase was added, statically maintained for 2-10 h, filtered and centrifuged to obtain a clear solution; sugar degree was adjusted to 6-10° Bx; and a pH was adjusted to 4-7.

A solid medium was prepared by wetting brans or corn flour with water and then steaming for 30 min.

Detection Method:

A flavor compound detection method for a liquid grain culture: A liquid culture was centrifuged to remove the microorganisms, and a supernatant was collected and subjected to headspace solid phase microextraction gas chromatography-mass spectrometry detection.

A flavor detection method for a solid sorghum culture: Ultrapure water was added to a solid culture, mixed uniformly by vibration and centrifuged. Supernatant was collected and subjected to headspace solid phase microextraction gas chromatography-mass spectrometry detection.

A chromatographic condition is shown in the paper: Peng Wang, Qun Wu, Xuejian Jiang, Zhiqiang Wang, Jingli Tang, Yan Xu. Bacillus licheniformis affects the microbial community and metabolic profile in the spontaneous fermentation of Daqu starter for Baijiu making. International Journal of Food Microbiology, 2017.

### Example 1: Separation and Screening of P. acidilactici from Fermented Grains of Baijiu

(1) Strain Separation and Screening

5 g of fermented grain sample was dissolved in 150 mL sterile saline solution, fully vibrated and uniformly mixed for 30 min, diluted 10⁷ times, then used to spreading on MRS solid medium with Triton, and cultured at 37° C. for 48 h; a single colony was selected; the selected single colony was transferred to an acid-producing screening medium; and a single colony with the medium obviously changing to yellow around the colony was selected. The selected single colony was transferred to a basic medium and cultured for 36 h, and a basic culture solution was transferred to a liquid grain medium for fermentation, and detection of flavor compounds was carried out as well.

(2) Strain Identification

The strain obtained by the screening in the step (1) was subjected to molecular biological identification, 16S rDNA fragments of the screened microorganisms were amplified by a bacterial classification identification primer, and gel electrophoresis was performed as well. An amplification product was sequenced and subjected to sequence alignment. The strain screened above was P. acidilactici, with its 16S rDNA as follows:

(SEQ ID NO. 1)
AATAATGCAGTCGAACGAACTTCCGTTAATTGATTATGAGGTGCTTGCAC
TGAATGAGATTTTAACACGAAGTGAGTGGCGGACGGGTGAGTAACACGTG
GGTAACCTGCCCAGAAGCAGGGGATAACACCTGGAAACAGATGCTAATAC
CGTATAACAGAGAAAACCGCCTGGTTTTCTTTTGAAAGATGGCTCTGCTA
TCACTTCTGGATGGACCCGCGGCGCATTAGCTAGTTGGTGAGGTAACGGC
TCACCAAGGCGATGATGCGTAGCCGACCTGAGAGGGTAATCGGCCACATT
GGGACTGAGACACGGCCCAGACTCCTACGGGAGGCAGCAGTAGGGAATCT
TCCACAATGGACGCAAGTCTGATGGAGCAACGCCGCGTGAGTGAAGAAGG
GTTTCGGCTCGTAAAGCTCTGTTGTTAAAGAAGAACGTGGGTGAGAGTAA
CTGTTCACCCAGTGACGGTATTTAACCAGAAAGCCACGGCTAACTACGTG
CCAGCAGCCGCGGTAATACGTAGGTGGCAAGCGTTATCCGGATTTATTGG
GCGTAAAGCGAGCGCAGGCGGTCTTTTAAGTCTAATGTGAAAGCCTTCGG
CTCAACCGAAGAAGTGCATTGGAAACTGGGAGACTTGAGTGCAGAAGAGG
ACAGTGGAACTCCATGTGTAGCGGTGAAATGCGTAGATATATGGAAGAAC
ACCAGTGGCGAAGGCGGCTGTCTGGTCTGTAACTGACGCTGAGGCTCGAA
AGCATGGGTAGCGAACAGGATTAGATACCCTGGTAGTCCATGCCGTAAAC
GATGATTACTAAGTGTTGGAGGGTTTCCGCCCTTCAGTGCTGCAGCTAAC
GCATTAAGTAATCCGCCTGGGGAGTACGACCGCAAGGTTGAAACTCAAAA
GAATTGACGGGGGCCCGCACAAGCGGTGGAGCATGTGGTTTAATTCGAAG
CTACGCGAAGAACCTTACCAGGTCTTGACATCTTCTGCCAACCTAAGAGA
TTAGGCGTTCCCTTCGGGGACAGAATGACAGGTGGTGCATGGTTGTCGTC
AGCTCGTGTCGTGAGATGTTGGGTTAAGTCCCGCAACGAGCGCAACCCTT
ATTACTAGTTGCCAGCATTCAGTTGGGCACTCTAGTGAGACTGCCGGTGA
CAAACCGGAGGAAGGTGGGGACGACGTCAAATCATCATGCCCCTTATGAC
CTGGGCTACACACGTGCTACAATGGATGGTACAACGAGTTGCGAAACCGC
GAGGTTTAGCTAATCTCTTAAAACCATTCTCAGTTCGGACTGTAGGCTGC
AACTCGCCTACACGAAGTCGGAATCGCTAGTAATCGCGGATCAGCATGCC
GCGGTGAATACGTTCCCGGGCCTTGTACACACCGCCCGTCACACCATGAG
AGTTTGTAACACCCAAAGCCGGTGGGGTAACCTTTAGGAGCTAGC.

### Example 2: Tolerance Detection of the Strain

(1) Acid Resistance Test of the Strain

A loop of the P. acidilactici strain screened in Example 1 was inoculated into a liquid basic medium and cultured for 28 h. The pH of the basic medium was adjusted to 5.0, 4.0, 3.0, 2.7, 2.4 and 2.1 with lactic acid or hydrochloric acid, respectively; each pH was provided with 3 groups of values in parallel; and a basic culture solution with OD₆₀₀=1 was added to mediums that were different in pH, and the solution was statically cultured at 37° C. for 48 h. Growth of the strain was shown in Table 1. Results showed that the P. acidilactici CGMCC NO. 22237 was resistant to acids, could adapt to a fermentation environment like fermented grains, and could play an important role in the fermentation system.

TABLE 1
Growth of the P. acidilactici CGMCC NO. 22237 under an Acidic Condition

| pH | 5.0 | 4.0 | 3.0 | 2.7 | 2.4 | 2.1 |
|---|---|---|---|---|---|---|
| OD₆₀₀ | 0.79-0.82 | 0.56-0.57 | 0.23-0.26 | 0.12-0.16 | 0.04-0.09 | 0-0.01 |

(2) Ethanol Resistance Test of the Strain

A loop of the P. acidilactici strain was inoculated into a basic medium and statically cultured at 37° C. for 28 h to obtain a seed solution. Before inoculation, ethanol was added to make sure that the basic medium has an ethanol concentration of 3, 6, 9 and 12 (%, v/v); and a seed solution with OD₆₀₀=1 was added to the same volume of mediums containing different concentrations of ethanol, and then the seed solution was statically cultured at 37° C. for 48 h. Growth of the strain was shown in Table 2. Results showed that the P. acidilactici CGMCC NO. 22237 was resistant to ethanol, could adapt to a fermentation environment like fermented grains, and could play an important role in the fermentation system.

TABLE 2
Growth of the P. acidilactici CGMCC NO. 22237
under a Condition with Ethanol (OD₆₀₀)

Concentration (%, v/v) of ethanol

| | 3 | 6 | 9 | 12 |
|---|---|---|---|---|
| OD₆₀₀ | 0.77-0.78 | 0.44-0.45 | 0.1-0.12 | 0-0.02 |

(3) High Temperature Resistance Test of the Strain

A circle of the P. acidilactici strain was inoculated into a basic medium and statically cultured at 37° C. for 28 h to obtain a seed solution. A 1% (v/v) seed solution with OD₆₀₀=1 was inoculated into the basic medium, and the same volume of culture was statically cultured at 25° C., 30° C., 37° C., 45° C. and 50° C. respectively for 48 h. Growth of the strain was shown in Table 3. Results showed that P. acidilactici CGMCC NO. 22237 was resistant to a high temperature, could adapt to a fermentation environment like fermented grains, and could play an important role in the fermentation system.

TABLE 3
Growth of the P. acidilactici CGMCC NO.
22237 at Different Temperatures (OD₆₀₀)

| | Temperature (° C.) | | | | |
|---|---|---|---|---|---|
| | 25 | 30 | 37 | 45 | 50 |
| OD₆₀₀ | 0.90-0.95 | 1.18-1.24 | 1.36-1.42 | 0.38-0.44 | 0.08-0.12 |

(4) High Salt Resistance Test of the Strain

A loop of the P. acidilactici strain was inoculated into a basic medium and statically cultured at 37° C. for 28 h to obtain a seed solution. A 1% (v/v) seed solution with OD₆₀₀=1 was inoculated into the basic medium, and the same volume of culture was statically cultured at 37° C. for 48 h in the basic mediums containing 50 g/L, 70 g/L, 90 g/L, 110 g/L and 130 g/L of NaCl, respectively. Growth of the strain was shown in Table 4. Results showed that the P. acidilactici CGMCC NO. 22237 was resistant to high salt, could adapt to a fermentation environment like fermented grains, and could play an important role in the fermentation system.

TABLE 4
Growth of the P. acidilactici CGMCC NO. 22237
under Different Salt Concentrations (OD₆₀₀)

| | Salt concentration (g/L) | | | | |
|---|---|---|---|---|---|
| | 3 | 5 | 7 | 9 | 11 |
| OD₆₀₀ | 0.79-0.84 | 0.54-0.59 | 0.16-0.21 | 0.05-0.08 | 0-0.01 |

### Example 3 Flavor Production Potential Analysis of the P. acidilactici CGMCC NO. 22237

(1) Sequencing of the Whole Genome of the Strain

The whole genome of the P. acidilactici CGMCC NO. 22237 was sequenced. A whole genome extraction method, a library construction method, a fragment purification method and a PacBio platform sequencing method refer to the following paper: Won Hyong Chung, Jisu Kang, Mi Young Lim, Tae-joong Lim, Sanghyun Lim, Seong Woon Roh and Young-Do Nam, 2018. Complete genome sequence and genomic characterization of Lactobacillus acidophilus LA1 (11869BP). Frontiers in pharmacology 9.

An assembled genome sequence of the P. acidilactici CGMCC NO. 22237 was compared with a reported P. acidilactici genome, and the information of the genome was shown in Table 5.

TABLE 5
Comparation of basic genomes of the P. acidilactici CGMCC NO. 22237

| | P. acidilactici CGMCC | P. acidilactici | P. acidilactici | P. acidilactici |
|---|---|---|---|---|
| | NO. 22237 | S1 | SRCM100320 | DSM 19927 |
| Genome Size (Mb) | 1.98 | 1.98 | 2.07 | 2.04 |
| GC Content (%) | 41.99 | 42.03 | 42.06 | 42.13 |
| rRNA | 15 | 7 | 12 | 2 |
| tRNA | 56 | 40 | 54 | 44 |
| Gene Number | 1886 | 2003 | 2017 | 1973 |

The corresponding situation of an NCBI Genbank assembly number of the P. acidilactici strain for comparative analysis was: P. acidilactici 51: GCA_001461015.1, coming from a rice wine brewing system; P. acidilactici SRCM100320: GCA_001672605.1, coming from pit mud; and P. acidilactici DSM 19927: GCA_001437115.1, coming from a bean paste fermentation system.

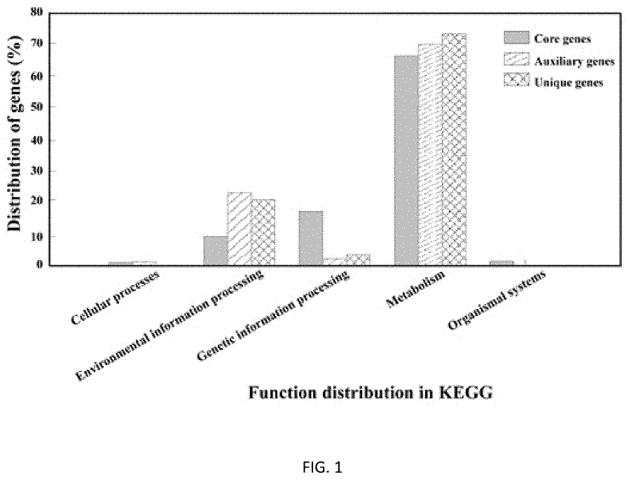

The P. acidilactici CGMCC NO. 22237 had a genome size of 1.98 Mb, a GC content of 41.99%, a gene number of 1886, and an average gene length of 1049.84 bp. The distribution of KEGG genes of the P. acidilactici CGMCC NO. 22237 was shown in FIG. 1. The P. acidilactici CGMCC NO. 22237 has unique genes, which all the other three P. acidilactici strains do not have. The P. acidilactici CGMCC NO. 22237 was annotated in the KEGG database, and its unique genes were distributed in the functions of environmental information processing, gene information processing and metabolism.

(2) Substrate Utilization and Flavor Compound Production Potential of the Strain

The unique genes for a metabolic function of the strain are closely related to flavor production, hence, the unique genes for the metabolic function were listed as shown in Table 6. The P. acidilactici CGMCC NO. 22237 has genes related to utilization of plurality of carbon sources, such as a gene for encoding carbohydrase including oligo-1,6-glucosidase. It can utilize carbon sources such as glucose, sucrose, galactose, lactose, melibiose, and rhamnose. The above substrate (20 g/L) was used as a single carbon source and cultured in a medium containing 15 g/L of KH₂PO₄, 2.5 g/L of NaCl, 33.9 g/L of Na₂HPO₄ and 5 g/L NH₄Cl, and the P. acidilactici CGMCC NO. 22237 could grow normally. Results showed that the strain could utilize a plurality of types of carbon sources, and could better improve substrate utilization rate, and increase the yield and diversity of flavor compound in food fermentation.

TABLE 6
Unique Genes for the Metabolic Function of the P. acidilactici
CGMCC NO. 22237 in the KEGG Database

| Gene ID | Metabolic Pathway | Enzyme | Function |
|---|---|---|---|
| K02791 | Glycolysis/Gluconeogenesis | EC: 2.7.1.199 | D-Glucose → α-6- |
| | Pathway | | Phosphoglucose |
| K01854 | Metabolic Pathway of | EC: 5.4.99.9 | Galactopyranose → |
| | Galactose | | Galactofuranose |
| K12308 | Metabolic Pathway of | EC: 3.2.1.23 | Lactose → D-Glucose + |
| | Galactose | | D-Galactose |
| K01235 | — | EC: 3.2.1.139 | α-Glucuronide → Ethanol + |
| | | | D-glucuronate |
| K01193 | Metabolic Pathway of | EC: 3.2.1.26 | Sucrose → D-Glucose + |
| | Starch and Sucrose | | D-Fructose |
| K01198 | Metabolic Pathway of | EC: 3.2.1.37 | D-Xylose Xylan |
| | Amino Sugar and | | |
| | Nucleotide Sugar | | |
| K07407 | Metabolic Pathway of | EC: 3.2.1.22 | Melibiose → D-Glucose + |
| | Galactose | | D-Galactose |
| K12996 | — | EC: 2.4.1.— | Utilization of β-L- |
| | | | Rhamnose |
| K00627 | Pentose Phosphate | EC: 2.3.1.12 | Pyruvate Dehydrogenation |
| | Pathway | | |
| K13954 | Degradation Pathway of | EC: 1.1.1.1 | Alcohols → Aldehydes |
| | Fatty Acid | | |

(3) Safety Evaluation of the P. acidilactici CGMCC NO. 22237

The synthesis pathways and enzymes for biogenic amines and nitrites closely related to the safety of fermented foods were searched in a KEGG pathway database and the results were shown in Table 7. The whole genome sequencing and annotation results showed that the P. acidilactici CGMCC NO. 22237 neither had metabolic pathway synthesizing biogenic amines nor had genes encoding enzymes related to the synthesis of biogenic amines. The P. acidilactici CGMCC NO. 22237 could be safely used in fermented foods because of its characteristic of non-production of biogenic amines and nitrites.

TABLE 7
Synthesis Pathways and Enzymes for Biogenic Amines and Nitrites

| Metabolic | | | |
|---|---|---|---|
| Pathway | Gene ID | Enzyme | Function |
| Degradation of | K01582 | EC: 4.1.1.18 | Generation of |
| Lysine | | | Cadaverine |
| | K23385 | EC: 4.1.1.116 | Generation of |
| | | | Cadaverine |
| Metabolism of | K01583, K01584, | EC: 4.1.1.19 | Arginine → |
| Arginine | K01585, K02626 | | Agmatine |
| and Proline | K01480 | EC: 3.5.3.11 | Agmatine → |
| | | | Putrescine |
| | K00797 | EC: 2.5.1.16 | Putrescine → |
| | | | Spermidine, |
| | | | Spermine |
| | K01476 | EC: 3.5.3.1 | Arginine → |
| | | | Ornithine |
| | K01581 | EC: 4.1.1.17 | Ornithine → |
| | | | Putrescine |
| Metabolism of | K01590 | EC: 4.1.1.22 | Histidine → |
| Histidine | | | Histamine |
| Metabolism of | k22329, k22330, | EC: 4.1.1.25 | Tyrosine → Tyramine |
| Tyrosine | k01592, k18933 | | |
| Metabolism of | K01593 | EC: 4.1.1.28 | Tryptophan → Tryptamine |
| Tryptophan | | EC1.7.1.1 EC 1.7.1.2 | Nitrate → Nitrite |
| | | EC 1.7.1.3 EC 1.7.5.1 | |
| | | EC 1.7.7.2 EC 1.9.6.1 | |

### Example 4: Preparation of a Solid Seed of the P. acidilactici CGMCC NO. 22237

A preparation method of a solid seed of the P. acidilactici CGMCC NO. 22237 includes the following steps: a loop of the P. acidilactici CGMCC NO. 22237 was inoculated into a liquid basic medium and statically cultured at 37° C. for 28 h to obtain a basic culture seed solution; the basic culture seed solution was inoculated into a liquid grain medium and statically cultured at 37° C. for 28 h to obtain a liquid grain culture solution with a strain concentration of at least 10⁸ CFU/mL; and the liquid grain culture solution was inoculated into a solid medium with an inoculum of 30% of the brans weight, uniformly mixed and cultured for 30-36 h to obtain a solid Fuqu seed. The solid Fuqu seed had a water content of 10% and a viable number ranging from 10⁸-10¹⁰ CFU/g.

### Example 5 Production of Flavor Compounds by the P. acidilactici CGMCC NO. 22237 in a Simulative Baijiu Fermentation System

The P. acidilactici CGMCC NO. 22237 was cultured in a basic medium according to the method in Example 1 to obtain a seed solution; and the seed solution with a final concentration of 10⁸ CFU/mL was inoculated into a liquid grain medium and statically cultured at 37° C. for 28 h, then flavor detection was performed, and 24 flavor compounds had been detected in a liquid culture of the P. acidilactici CGMCC NO. 22237.

The solid Fuqu seed mentioned in Example 4 was inoculated into a steamed sorghum substrate, uniformly mixed and hermetically cultured for 7 d, and then flavor detection was performed.

The sorghum substrate was prepared as follows: sorghum was soaked in ultrapure water for 14 h, which was 3 times the weight of the sorghum; 30 U/g of α-thermostable amylase was added, uniformly mixed and cooked for 60 min; a content of reducing sugar was 70 g/kg; the sorghum was used as a substrate for inoculating a solid inoculant; and the solid inoculant was 5% the weight of the sorghum substrate.

There were 22 volatile flavor compounds detected in a solid culture, including key flavor compounds of Baijiu, such as isobutanol, isoamylol, 2,3-butanediol, phenylethanol, acetic acid, isovaleric acid, valeric acid, caproic acid, caprylic acid, ethyl acetate, ethyl valerate, ethyl hexanoate, ethyl caprylate, nonanal, 3-hydroxy-2-butanone, and 2,4-ditert-butylphenol. Types and yields of the volatile flavor compounds were shown in Table 8.

TABLE 8
Flavor Characteristics of the Strain (μg/L or μg/kg)

| | P. acidilactici CGMCC NO. 22237 | | Other P. acidilactici Strains | |
|---|---|---|---|---|
| Flavor | Liquid | Solid | Liquid | Solid |
| Compounds | Fermentation | Fermentation | Fermentation | Fermentation |
| Isobutanol | 50-80 | 50-60 | 50-80 | 50-80 |
| Isoamylol | 80-120 | 80-120 | 80-100 | 80-100 |
| 2,3-Butanediol | 20-40 | 20-40 | 10-30 | 20-30 |
| 1-Hexanol | 20-30 | 10-20 | 10-30 | 10-30 |
| 1-Heptanol | 10-30 | 10-20 | 20-30 | 20-30 |
| 2-Ethyl Hexanol | 5-10 | 0 | 1-3 | 0 |
| Phenylethanol | 150-220 | 150-220 | 100-120 | 80-100 |
| Acetic Acid | 50-80 | 50-100 | 40-60 | 40-60 |
| Valeric Acid | 10-20 | 10-20 | 5-20 | 5-15 |
| Caproic Acid | 60-80 | 40-60 | 50-60 | 30-50 |
| Caprylic Acid | 50-80 | 60-90 | 50-70 | 50-70 |
| Nonanoic Acid | 10-20 | 10-15 | 1-5 | 0 |
| N-decanoic Acid | 20-30 | 10-20 | 1-5 | 0 |
| Ethyl Acetate | 30-50 | 40-60 | 30-60 | 30-60 |
| Isoamyl Acetate | 40-50 | 40-50 | 30-50 | 30-40 |
| Ethyl Valerate | 20-30 | 10-20 | 10-20 | 10-20 |
| Ethyl Hexanoate | 30-60 | 30-50 | 30-40 | 20-40 |
| Ethyl Caprylate | 40-60 | 60-70 | 50-60 | 40-50 |
| Ethyl Nonylate | 5-10 | 0 | 5-10 | 0 |
| Ethyl Caprate | 60-80 | 50-80 | 5-10 | 0-5 |
| Phenethyl Acetate | 30-50 | 60-70 | 20-40 | 30-40 |
| Nonanal | 40-60 | 40-60 | 10-50 | 10-50 |
| 3-Hydroxy-2- | 20-40 | 20-40 | 10-30 | 10-30 |
| Butanone | | | | |
| 2,4-Di-Tert- | 50-100 | 50-100 | 20-60 | 20-50 |
| Butylphenol | | | | |

### Example 6: Application of the P. acidilactici CGMCC NO. 22237 in Promoting Other Microorganisms to Produce Flavor Compounds

(1) Application of the P. acidilactici CGMCC NO. 22237 in Promoting S. cerevisiae to Produce Flavor Compounds

A preparation method of a S. cerevisiae solid inoculant includes the following steps: A loop of S. cerevisiae was inoculated into a liquid basic medium, shaken at 30° C. for 24 h to obtain a basic culture seed solution; the basic culture seed solution was inoculated into a liquid grain medium, shaken at 30° C. for 24 h to obtain a liquid grain culture solution with a strain concentration equal to or higher than 10⁸ CFU/mL; and the liquid grain culture solution which was 30% of the weight of brans was inoculated into a solid bran medium, uniformly mixed and cultured for 48 h to obtain a solid Fuqu. The solid Fuqu had a water content of 10%, and the S. cerevisiae solid inoculant had a viable number of at least 10⁹ CFU/g.

Sorghum was steamed under a condition as follows: The sorghum was soaked in ultrapure water for 14 h, which was 2 times of the weight of the sorghum and steamed for 60 min to obtain a material used as a substrate for inoculating starter culture or a solid inoculant, and the solid inoculant or the was 5% of the weight of the sorghum substrate.

The prepared S. cerevisiae solid inoculant was inoculated into the steamed sorghum, with the P. acidilactici CGMCC NO. 22237 solid inoculant, and a viable strain ratio of the S. cerevisiae to the P. acidilactici CGMCC NO. 22237 was 10⁶ CFU/g:10⁵ CFU/g; and they were uniformly mixed and hermetically fermented at 30° C. for 7 d, and then flavor detection was performed.

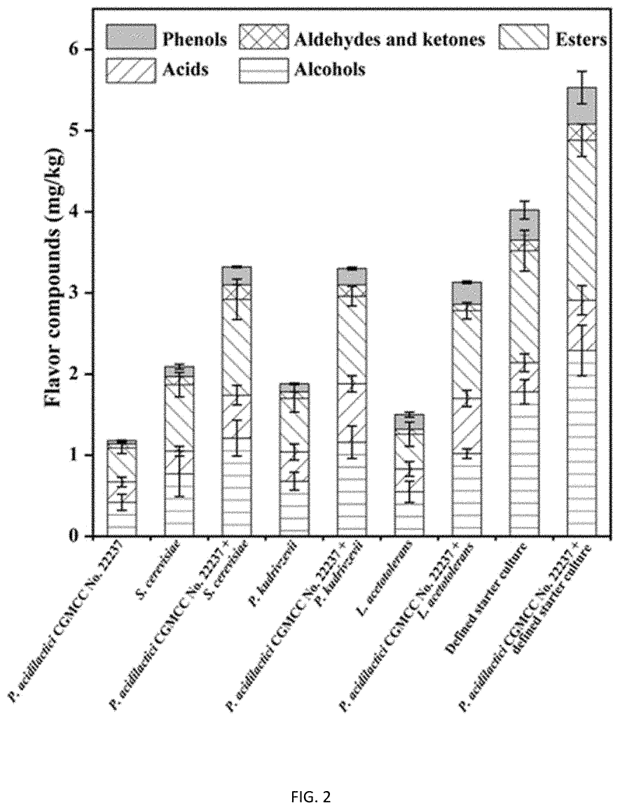

When fermented independently, the S. cerevisiae solid inoculant produced 26 flavor compounds, including 22 flavor compounds produced by the P. acidilactici CGMCC NO. 22237 solid inoculant in independent fermentation; and the S. cerevisiae solid inoculant and the P. acidilactici CGMCC NO. 22237 solid inoculant produced 28 flavor compounds by co-fermentation, including the 26 flavor compounds produced by the S. cerevisiae solid inoculant in independent fermentation. Content of volatile flavor compounds was shown in FIG. 2. A total amount of the flavor compounds produced in independent inoculation and fermentation of the S. cerevisiae solid inoculant was 2.09 mg/kg; the total amount of the flavor compounds produced in independent inoculation and fermentation of the P. acidilactici solid inoculant was 1.19 mg/kg; a yield of the flavor compounds produced in co-fermentation of the P. acidilactici solid inoculant and the S. cerevisiae solid inoculant was 3.36 mg/kg; and the yield of the flavor compounds produced in co-cultivation was higher than a sum of the yields of the flavor compounds that were produced in independent cultivation. Results indicated that the P. acidilactici CGMCC NO. 22237 could promote the metabolic characteristics of the S. cerevisiae to improve the metabolic performance of the S. cerevisiae, thereby achieving a higher yield and producing more types of flavor compounds. A better flavor compound production capability was achieved when the P. acidilactici and the S. cerevisiae were co-cultured.

(2) Application of the P. acidilactici CGMCC NO. 22237 in Promoting P. kudriavzevii to Produce Flavor Compounds

A preparation method of a P. kudriavzevii solid inoculant includes the following steps: A loop of a P. kudriavzevii inoculant was inoculated into a liquid basic medium, shaken at 30° C. for 24 h to obtain a basic culture seed solution; the basic culture seed solution was inoculated into a liquid grain medium, shaken at 30° C. for 24 h to obtain a liquid grain culture solution with a strain concentration equal to or higher than 10⁸ CFU/mL; and the liquid grain culture solution, which is 30% of the weight of brans was inoculated into a solid bran medium, uniformly mixed and cultured for 48 h to obtain a solid Fuqu inoculant. The solid Fuqu inoculant had a water content of 10%, and the P. kudriavzevii solid inoculant had a viable number of at least 10⁹ CFU/g.

Sorghum was steamed under a condition as follows: The sorghum was soaked in ultrapure water which was 2 times the weight of the sorghum for 14 h and steamed for 60 min to obtain a material used as a substrate for inoculating starter culture or a solid inoculant, and the solid inoculant or the starter culture were 5% of the weight of the sorghum substrate.

The prepared P. kudriavzevii solid inoculant was inoculated into the steamed sorghum, and was also inoculated into the steamed sorghum substrate together with the P. acidilactici CGMCC NO. 22237 solid inoculant, and a viable strain ratio of the P. kudriavzevii to the P. acidilactici CGMCC NO. 22237 was 10⁶ CFU/g:10⁵ CFU/g; and they were uniformly mixed and hermetically fermented at 30° C. for 7 d, and then flavor detection was performed.

When fermented independently, the P. kudriavzevii solid inoculant produced 23 flavor compounds, including 22 flavor compounds produced by the P. acidilactici CGMCC NO. 22237 solid inoculant in independent fermentation; and the P. kudriavzevii and the P. acidilactici CGMCC NO. 22237 solid inoculant produced 25 flavor compounds by co-fermentation, including the 23 flavor compounds produced by the P. kudriavzevii in independent fermentation. Content of volatile flavor compounds was shown in FIG. 2. A total amount of the flavor compounds produced in independent inoculation and fermentation of the P. kudriavzevii solid inoculant was 1.88 mg/kg; the total amount of the flavor compounds produced in independent inoculation and fermentation of the P. acidilactici solid inoculant was 1.19 mg/kg; a yield of the flavor compounds produced in co-fermentation of the P. acidilactici inoculant and the P. kudriavzevii inoculant was 3.30 mg/kg; and the yield of the flavor compounds produced in co-cultivation was higher than the sum of the yields of the flavor compounds that were produced by independent cultivation. Results indicated that the P. acidilactici CGMCC NO. 22237 could regulate the metabolic characteristics of the P. kudriavzevii to improve the metabolic performance of the P. kudriavzevii, thereby achieving a higher yield and producing more types of flavor compounds. A better flavor production capability was achieved when the P. acidilactici and the P. kudriavzevii were co-cultured.

(3) Application of the P. acidilactici CGMCC NO. 22237 in Promoting L. scetotolerans to Produce Flavor Compounds

A preparation method of a L. acetotolerans solid inoculant includes the following steps: A loop of a L. acetotolerans inoculant was inoculated into a liquid basic medium, statically cultured at 37° C. for 72 h to obtain a basic culture seed solution; the basic culture seed solution was inoculated into a liquid grain medium, statically cultured at 37° C. for 48 h to obtain a liquid grain culture solution with a strain concentration equal to or higher than 10⁸ CFU/mL; and the liquid grain culture solution which was 35% of the weight of brans was inoculated into a solid bran medium, uniformly mixed and cultured for 48 h to obtain a solid bran koji inoculant. The solid bran koji inoculant had a water content of 10%, and the L. acetotolerans solid inoculant had a viable number of 10⁸-10¹⁰ CFU/g.

Sorghum was steamed under a condition as follows: The sorghum was soaked in ultrapure water with a weight 2 times of the sorghum for 14 h and steamed for 60 min to obtain a material used as a substrate for inoculating a solid inoculant or starter culture, which was 5% the weight of the sorghum substrate.

The prepared L. acetotolerans solid inoculant was inoculated into the steamed sorghum, and was also inoculated into the steamed sorghum substrate together with the P. acidilactici CGMCC NO. 22237 solid inoculant, and a viable strain ratio of the L. acetotolerans to the P. acidilactici CGMCC NO. 22237 was controlled to be 10⁶ CFU/g:10⁵ CFU/g; and they were uniformly mixed and hermetically fermented at 30° C. for 7 d, and then flavor detection was performed.

When fermented independently, the L. acetotolerans solid inoculant produced 24 flavor compounds, including 22 flavor compounds produced by the P. acidilactici CGMCC NO. 22237 solid inoculant in independent fermentation; and the L. acetotolerans and the P. acidilactici CGMCC NO. 22237 solid inoculant produced 26 flavor compounds by co-fermentation, including the 24 flavor compounds produced by the L. acetotolerans in independent fermentation. Content of volatile flavor compounds was shown in FIG. 2. A total amount of the flavor compounds produced in independent inoculation and fermentation of the L. acetotolerans solid inoculant was 1.50 mg/kg; the total amount of the flavor compounds produced in independent inoculation and fermentation of the P. acidilactici inoculant was 1.19 mg/kg; a yield of the flavor compounds produced in co-fermentation of the P. acidilactici inoculant and the L. acetotolerans inoculant was 3.13 mg/kg; and the yield of the flavor compounds produced in co-cultivation was higher than a sum of the yields of the flavor compounds that were produced in independent cultivation. Results indicated that the P. acidilactici CGMCC NO. 22237 could regulate the metabolic characteristics of the L. acetotolerans to improve the metabolic performance of the L. acetotolerans, thereby achieving a higher yield and producing more types of flavor compounds. A better flavor production capability was achieved when the P. acidilactici CGMCC and the L. acetotolerans were co-cultured.

(4) Application of the P. acidilactici CGMCC NO. 22237 in Promoting Flavor-Producing Microbial Floras to Produce Flavor Compounds

A solid inoculant consisting of important flavor-producing microorganisms of Baijiu includes: Rhizopus chinensis, S. cerevisiae, P. kudriavzevii, Wickerhamomyces anomalus, and L. acetotolerans, and was inoculated in steamed sorghum; and the P. acidilactici CGMCC NO. 22237 and the above flavor-producing microorganism combined inoculant were co-inoculated into the steamed sorghum. They were cultured under an aerobic condition at 30° C. for 24 h, and then hermetically fermented at 30° C. for 7 d.

A preparation method of a combined inoculant includes the following steps: (1) a Rhizopus inoculant: a loop of a R. chinensis was inoculated into a spore culture medium, at 30° C. for 72 h and then eluted with a buffer to remove spores to obtain a spore solution; the spore solution was inoculated into a solid Rhizopus medium and cultured at 30° C. for 36 h to obtain a R. chinensis solid inoculant; (2) a Saccharomyces inoculant: a loop of Saccharomyces was inoculated in a liquid basic medium, shaken at 30° C. for 24 h to prepare a culture solution; the culture solution was transferred to a liquid grain medium, cultivation at 30° C. for 24 h to obtain a seed solution; the prepared seed solution was inoculated into a solid medium and statically cultured at 30° C. for 24 h to obtain a Saccharomyces solid inoculant; and (3) a Lactobacillus inoculant: a loop of Lactobacillus was inoculated into a liquid basic medium and cultured at 37° C. for 72 h to obtain a culture solution; the culture solution was transferred to a liquid grain medium and cultured at 37° C. for 72 h to obtain a seed solution; the seed solution was inoculated into a solid medium and cultured at 37° C. for 72 h to obtain Lactobacillus solid inoculant. The Rhizopus inoculant, the Saccharomyces inoculant and the Lactobacillus inoculant were mixed, and the R. chinensis, the S. cerevisiae, the P. kudriavzevii, the W. anomalus and the L. acetotolerans were mixed according to a viable number ratio of 1:1:1:1:1 to prepare the combined starter culture. A total viable number of the starter culture: P. acidilactici CGMCC NO. 22237 inoculant is 10⁷ CFU/g:10⁵ CFU/g.

Sorghum was steamed under a condition as follows: The sorghum was soaked in ultrapure water with a weight 2 times of the sorghum for 14 h and steamed for 60 min, and used as a substrate for inoculating the combined solid inoculant or a mixture of the P. acidilactici CGMCC NO. 22237 inoculant and the combined solid inoculant, and the solid inoculant was 5% of the weight of the sorghum substrate.

After fermentation of the inoculated combined inoculant was ended, 52 flavor compounds were detected, including the 22 flavor compounds produced in independent fermentation of the P. acidilactici CGMCC NO. 22237 solid inoculant, with a total amount of 3.67 mg/kg; after fermentation of the inoculated combined inoculant and the P. acidilactici CGMCC NO. 22237 inoculant was ended, 58 flavor compounds were detected, including the 52 flavor compounds produced in fermentation of the combined inoculant, and there was a total amount of 5.53 mg/kg of the produced flavor compounds. Results indicated that the P. acidilactici CGMCC NO. 22237 could regulate the metabolic characteristics of the floras to improve the metabolic performance of the floras, thereby achieving a higher yield and producing more types of flavor compounds. A better flavor production capability was achieved by adding the P. acidilactici solid inoculant.

### Implementation 7: Application of the P. acidilactici CGMCC NO. 22237 in Enhancing Production of Flavor Compounds of Baijiu

In an initial stage of Baijiu fermentation, starter culture which was 2% of the weight of grains was inoculated together with 0.2% of a P. acidilactici CGMCC NO. 22237 solid Fuqu inoculant, and was fermented for 15-60 d according to production processes of Baijiu with different flavors, and distilled in a retort; the liquor was collected; flavor compounds of the liquor were detected; and contents of the flavor compounds with different starting conditions were shown in Table 9.

TABLE 9
Application Effects of the P. acidilactici CGMCC NO. 22237 in Baijiu

| | | | | | Aldehydes | |
|---|---|---|---|---|---|---|
| | | Alcohols | Acids | Esters | and Ketones | Phenols |
| Addition of the | Numbers of Volatile | 16 | 8 | 21 | 5 | 8 |
| P. acidilactici | Flavor Compound | | | | | |
| CGMCC NO. | Types | | | | | |
| 22237 | Content (mg/L) of | 1.5-1.8 | 0.55-0.7 | 0.85-1.2 | 0.15-0.25 | 0.07-0.1 |
| Inoculant | Volatile Flavor | | | | | |
| | Compounds | | | | | |
| Addition of | Numbers of Volatile | 15 | 6 | 20 | 3 | 6 |
| Other | Flavor Compound Type | | | | | |
| P. acidilactici | Content (mg/L) of | 1.5-1.6 | 0.4-0.5 | 0.8-0.9 | 0.12-0.2 | 0.06-0.08 |
| | Volatile Flavor | | | | | |
| | Substances | | | | | |
| No Addition of | Volatile Flavor | 15 | 5 | 18 | 3 | 5 |
| Inoculants | Compound Types | | | | | |
| | Content (mg/L) of | 1.5-1.6 | 0.4-0.5 | 0.7-0.8 | 0.1-0.18 | 0.05-0.08 |
| | Volatile Flavor | | | | | |
| | Compounds | | | | | |

### Example 8: Application of the P. acidilactici CGMCC NO. 22237 in Fermentation of Other Foods

(1) Application in Fermented Pickles

A P. acidilactici CGMCC NO. 22237 solid inoculant which was 0.5% of the weight of substrate was added to an initial fermentation system of naturally fermented radish or cabbage pickles, and fermented at 12° C. for 30 d according to a natural pickle fermentation process.

(2) Application in Fermentation of Fruit and Vegetable Enzymes

A P. acidilactici CGMCC NO. 22237 solid inoculant which was 8% of the weight of substrate was added to the fruit and vegetable pulp, fermented at 30° C. for 7 d according to an enzyme fermentation process, and stirred at 150 r/min. Compared with a fermentation period of natural fermentation, the fermentation period of an enzyme product added with an inoculant was shortened by 16 h. Based on a volume ratio, the fruit and vegetable pulp includes 50% of apple pulp, 30% of pear pulp, and 20% of cucumber pulp, asparagus pulp or other vegetable pulp.

(3) Application in Fermented Sausages

P. acidilactici CGMCC NO. 22237 which was 0.2% of the weight of substrate was added to an initial fermentation system of naturally fermented sausages, and fermented at 20° C. for 5 d according to a natural sausage fermentation process.

The characteristics of the pickles, enzymes, and fermented meat products (taking sausages as an example) were shown in Table 10. A miscellaneous bacteria refer to Gram-negative microorganisms such as Escherichia coli, Acinetobacter, Vibrio and Proteus that are detrimental to food fermentation, human health or flavor production. Cells were stained using a crystalline violet solution with an iodine solution, decolorized using 95% ethanol, and stained again using a fuchsin solution. After such treatment, the microorganisms stained red when viewed through a microscope were Gram-negative bacteria. A hemocytometer was used for counting.

TABLE 10
Comparison of after-fermentation characteristics of foods
with and without the P. Acidilactici CGMCC NO. 22237

| | | | | Number (CFU/g) of |
|---|---|---|---|---|
| | | | | Miscellaneous |
| | Appearance | Flavor | Taste | Bacteria |
| Pickles with | Clear and | With Unique | Crisp and | 2 × 10²-4 × 10² |
| Inoculant | Bright Soup | Flavor | Tasty | |
| Pickles without | With a | With an | Soft | 0.5 × 10⁴-4 × 10⁴ |
| Inoculant | Floating Film | Undesirable | | |
| | | Flavor | | |
| Enzyme with | With Good | With Unique | Smooth in | 0.2 × 10²-1 × 10² |
| Inoculant | Transparency | Flavor | Taste | |
| | and | | | |
| | Uniformity | | | |
| Enzyme without | Relatively | Insufficient | Slightly Bitter | 1 × 10⁴-4 × 10⁴ |
| Inoculant | Muddy | Flavor | | |
| Sausage with | Rosy and | With Unique | Soft and Waxy | 0.5 × 10³-1 × 10³ |
| Inoculant | Tender | Flavor | | |
| Sausage without | Dark Red | With Peculiar | Hard in Taste | 2 × 10⁴-8 × 10⁴ |
| Inoculant | | Smell | | |

Although the disclosure has been disclosed above with preferred examples, the examples are not intended to limit the disclosure. Any person familiar with this art can make various changes and modifications without departing from the spirit and scope of the disclosure. Therefore, the scope of protection of the disclosure should be as defined in the claims.

What is claimed is:

1. A microbial preparation, comprising Pediococcus acidilactici (P. acidilactici) CGMCC NO. 22237, wherein the P. acidilactici CGMCC NO. 22237 was deposited in China General Microbiological Culture Collection Center on Apr. 25, 2021, at an address of 3 #, No. 1 Courtyard, Beichen West Road, Chaoyang District, Beijing, with an accession number of CGMCC NO. 22237.

2. The microbial preparation according to claim 1, wherein the microbial preparation is liquid or solid, and the P. acidilactici has a viable content ranging from 10⁸-10¹⁰ CFU/mL or CFU/g.

3. The microbial preparation according to claim 2, wherein the microbial preparation further contains a strain of a species allowed to be applied to foods or food preparation.

4. The microbial preparation according to claim 2, wherein the microbial preparation is Maiqu, Fuqu, Jiuyao, or other types of microbial inoculants prepared by using the P. acidilactici CGMCC NO. 22237.

5. The microbial preparation according to claim 4, wherein the microbial preparation is a solid inoculant containing the P. acidilactici CGMCC NO. 22237, and a preparation method of the microbial preparation comprises inoculating a bacterial solution containing the P. acidilactici CGMCC NO. 22237 into a solid medium and culturing for 30-48 hours to obtain a solid inoculant with a water content of 5-10% and a viable number of 10⁸-10¹⁰ CFU/g, and the solid medium is prepared by wetting bran or corn flour with water and then steaming them for 20-40 min.

6. A method for co-culturing the P. acidilactici CGMCC NO. 22237 with S. cerevisiae, P. kudriavzevii, L. acetotolerans or a complex starter culture, wherein in the co-culturing, the P. acidilactici CGMCC NO. 22237 is cultured after being mixed with the S. cerevisiae, the P. kudriavzevii or the L. acetotolerans inoculants at a ratio of (10⁵-10⁶) CFU/g:(10⁶-10⁷) CFU/g; the P. acidilactici CGMCC NO. 22237 and the complex starter culture are cultured after being mixed according to a ratio of (10⁵-10⁶) CFU/g:(10⁶-10⁷) CFU/g; in the co-culturing, sealed fermentation is performed at 28-37° C. for 5-7 days; and the P. acidilactici CGMCC NO. 22237 was deposited in China General Microbiological Culture Collection Center on Apr. 25, 2021, addressing at 3 #, No. 1 Courtyard, Beichen West Road, Chaoyang District, Beijing, with a collection number of CGMCC NO. 22237.

7. An application of the microbial preparation according to claim 1 in improving flavor compound characteristics and quality of Baijiu, wherein the microbial preparation is added to a Baijiu fermentation system.

8. The application according to claim 7, wherein the application is to improve at least one of the characteristics (1)-(6):
(1) a yield of characteristic flavor compounds of a food;
(2) metabolic diversity and quality of food flavors;
(3) food safety;
(4) substrate utilization;
(5) reduction of the yield of biogenic amine in the food; and
(6) reduction of the yield of nitrites in the food.


SEQUENCE LISTING (1 sequence)

SEQ ID NO: 1 - DNA, 1445 bp, Pediococcus acidilactici
  source 1..1445: mol_type = genomic DNA
aataatgcagtcgaacgaacttccgttaattgattatgaggtgcttgcactgaatgagattttaacacgaagtgagtggcggacgggtgagtaacacgtgggtaacctgcccagaagcaggggataacacctggaaacagatgctaataccgtataacagagaaaaccgcctggttttcttttgaaagatggctctgctatcacttctggatggacccgcggcgcattagctagttggtgaggtaacggctcaccaaggcgatgatgcgtagccgacctgagagggtaatcggccacattgggactgagacacggcccagactcctacgggaggcagcagtagggaatcttccacaatggacgcaagtctgatggagcaacgccgcgtgagtgaagaagggtttcggctcgtaaagctctgttgttaaagaagaacgtgggtgagagtaactgttcacccagtgacggtatttaaccagaaagccacggctaactacgtgccagcagccgcggtaatacgtaggtggcaagcgttatccggatttattgggcgtaaagcgagcgcaggcggtcttttaagtctaatgtgaaagccttcggctcaaccgaagaagtgcattggaaactgggagacttgagtgcagaagaggacagtggaactccatgtgtagcggtgaaatgcgtagatatatggaagaacaccagtggcgaaggcggctgtctggtctgtaactgacgctgaggctcgaaagcatgggtagcgaacaggattagataccctggtagtccatgccgtaaacgatgattactaagtgttggagggtttccgcccttcagtgctgcagctaacgcattaagtaatccgcctggggagtacgaccgcaaggttgaaactcaaaagaattgacgggggcccgcacaagcggtggagcatgtggtttaattcgaagctacgcgaagaaccttaccaggtcttgacatcttctgccaacctaagagattaggcgttcccttcggggacagaatgacaggtggtgcatggttgtcgtcagctcgtgtcgtgagatgttgggttaagtcccgcaacgagcgcaacccttattactagttgccagcattcagttgggcactctagtgagactgccggtgacaaaccggaggaaggtggggacgacgtcaaatcatcatgccccttatgacctgggctacacacgtgctacaatggatggtacaacgagttgcgaaaccgcgaggtttagctaatctcttaaaaccattctcagttcggactgtaggctgcaactcgcctacacgaagtcggaatcgctagtaatcgcggatcagcatgccgcggtgaatacgttcccgggccttgtacacaccgcccgtcacaccatgagagtttgtaacacccaaagccggtggggtaacctttaggagctagc